(19) United States
(12) Patent Application Publication
(10) Pub. No.: US 2023/0226041 A1
(43) Pub. Date: Jul. 20, 2023

(54) COMPOUNDS FOR THE TREATMENT OF VIRAL INFECTIONS

(71) Applicant: Merck Patent GmbH, Darmstadt (DE)

(72) Inventor: Ulrich BETZ, Darmstadt (DE)

(73) Assignee: Merck Patent GmbH, Darmstadt (DE)

(21) Appl. No.: 18/001,859

(22) PCT Filed: Jun. 15, 2021
(86) PCT No.: PCT/EP2021/066020

§ 371 Date: Dec. 14, 2022

(30) Foreign Application Priority Data
Jun. 18, 2020 (EP) 20180884.7

(51) Int. Cl. A61K 31/4745; A61K 45/06; A61P 31/14; A61K 31/706

(52) U.S. Cl. CPC A61K 31/4745; A61K 45/06; A61P 31/14; A61K 31/706

(57) ABSTRACT

An ATM inhibitor can be used in the treatment of coronavirus infections, including COVID-19, alone or in combination with one or more additional therapeutic agents.

### TECHNICAL FIELD OF THE INVENTION

The present invention provides for the use of ATM (ataxia telangiectasia mutated) inhibitors in the treatment of coronavirus infections, including SARS-CoV infections such as COVID-19.

### BACKGROUND OF THE INVENTION

### ATM Inhibitors

The serine/threonine protein kinase ATM (ataxia telangiectasia mutated kinase) belongs to the PIKK family of kinases having catalytic domains which are homologous with phospho-inositide-3 kinases (PI3 kinase, PI3K). These kinases are involved in a multiplicity of key cellular functions, such as cell growth, cell proliferation, migration, differentiation, survival and cell adhesion. In particular, these kinases react to DNA damage by activation of the cell cycle arrest and DNA repair programmes (DDR: DNA damage response). ATM is a product of the ATM gene and plays a key role in the repair of damage to the DNA double strand (DSB: double strand breaks). Double-strand damage of this type is particularly cytotoxic. ATM inhibitors are being developed for the treatment of cancer, in particular in combination with radiotherapy or in combination with other anticancer agents.

### Coronaviruses

Coronaviruses (CoVs) are positive-sense, single-stranded RNA (ssRNA) viruses of the order Nidovirales, in the family Coronaviridae. There are four sub-types of coronaviruses—alpha, beta, gamma and delta—with the Alphacoronaviruses and Betacoronaviruses infecting mostly mammals, including humans. Over the last two decades, three significant novel coronaviruses have emerged which jumped from a non-human mammal host to infect humans: the severe acute respiratory syndrome (SARS-CoV-1) which appeared in 2002, Middle East respiratory syndrome (MERS-CoV) which appeared in 2012, and COVID-19 (SARS-CoV-2) which appeared in late 2019. By mid-June of 2020, over 7.8 million people are known to have been infected, and over 432,000 people have died. Both numbers likely represent a significant undercount of the devastation wrought by the disease.

### COVID-19

SARS-CoV-2 closely resembles SARS-CoV-1, the causative agent of SARS epidemic of 2002-03 (Fung, et al, Annu. Rev. Microbiol. 2019. 73:529-57). Severe disease has been reported in approximately 15% of patients infected with SARS-CoV-2, of which one third progress to critical disease (e.g. respiratory failure, shock, or multiorgan dysfunction (Siddiqi, et al, J. Heart and Lung Trans. (2020), doi: https://doi.org/10.1016/j.healun.2020.03.012, Zhou, et al, Lancet 2020; 395: 1054-62. https://doi.org/10.1016/S0140-6736(20)30566-3). Fully understanding the mechanism of viral pathogenesis and immune responses triggered by SARS-CoV-2 would be extremely important in rational design of therapeutic interventions beyond antiviral treatments and supportive care. Much is still being discovered about the various ways that COVID-19 impacts the health of the people that develop it.

Severe acute respiratory syndrome (SARS)-Corona Virus-2 (CoV-2), the etiologic agent for coronavirus disease 2019 (COVID-19), has caused a pandemic affecting almost eight million people worldwide with a case fatality rate of 2-4% as of June 2020. The virus has a high transmission rate, likely linked to high early viral loads and lack of pre-existing immunity (He, et. al, Nat Med 2020 https://doi.org/10.1038/s41591-020-0869-5). It causes severe disease especially in the elderly and in individuals with comorbidities. The global burden of COVID-19 is immense, and therapeutic approaches are increasingly necessary to tackle the disease. Intuitive anti-viral approaches including those developed for enveloped RNA viruses like HIV-1 (lopinavir plus ritonavir) and Ebola virus (remdesivir) have been implemented in testing as investigational drugs (Grein et al, NEJM 2020 https://doi.org/10.1056/NEJMoa2007016; Cao, et al, NEJM 2020 DOI: 10.1056/NEJMoa2001282). But given that many patients with severe disease present with immunopathology, host-directed immunomodulatory approaches are also being considered, either in a staged approach or concomitantly with antivirals (Metha, et al, The Lancet 2020; 395(10229) DOI: https://doi.org/10.1016/S0140-6736(20)30628-0, Stebbing, et al, Lancet Infect Dis 2020. https://doi.org/10.1016/S1473-3099(20)30132-8).

While there are many therapies being considered for use in treatment of COVID-19, there are as yet no approved medications to treat the disease, and no vaccine is available. To date, treatment typically consists only of the available clinical mainstays of symptomatic management, oxygen therapy, with mechanical ventilation for patients with respiratory failure. Thus, there is an urgent need for novel therapies to address the different stages of the SARS-CoV-2 infectious cycle (Siddiqi, et al.).

### BRIEF DESCRIPTION OF THE DRAWINGS

FIG. 1 shows a graph depicting the confluence of Calu-3 cells when treated with concentrations between 4 and 27 μM of a first ATM inhibitor (“NCE4”) of the invention as compared to uninfected cells and infected cells without exposure to the therapeutic agent.

FIG. 2 shows a graph depicting the confluence of Calu-3 cells when treated with concentrations between 16 and 81 μM of a second ATM inhibitor (“NCE16”) of the invention as compared to uninfected cells and infected cells without exposure to the therapeutic agent.

### SUMMARY OF THE INVENTION

In a first embodiment, the invention provides ATM inhibitors of the invention for use in the treatment of viral infections in a subject in need thereof. In one aspect of this embodiment, the viral infection is a single-strand RNA viral infection. In another aspect of this embodiment, the viral infection is a coronavirus infection. In a further aspect of this embodiment, the viral infection is a SARS-CoV1, MERS-CoV, or SARS-CoV-2 infection. In a final aspect of this embodiment, the viral infection is a SARS-CoV-2 infection.

A second embodiment is a method of treating a coronavirus infection in a subject in need thereof, comprising administering an effective amount of an ATM inhibitor, or a pharmaceutically acceptable salt thereof, to the subject. In one aspect of this embodiment, the administration of the ATM inhibitor reduces the viral load in the subject. In one aspect of this embodiment, the ATM inhibitor is administered prior to COVID-19 pneumonia development. In another aspect of this embodiment, the ATM inhibitor is administered prior to the subject developing a severe cytokine storm. In a further aspect of this embodiment, the subject has a mild to moderate SARS-CoV-2 infection. In an additional aspect of this embodiment, the subject is asymptomatic at the start of the administration regimen.

### DETAILED DESCRIPTION

Recent papers have suggested a correlation between SARS-CoV-2 viral load, symptom severity and viral shedding (He, et al; Liu, et al, Lancet Infect Dis 2020. https://doi.org/10.1016/S1473-3099(20)30232-2). Some antiviral drugs administered at symptom onset to blunt coronavirus replication are in the testing phase (Grein, et al; Taccone, et al), but as yet none have shown much promise. Being able to slow the viral reproduction in the early stages of infection may allow the subject to avoid severe disease.

Coronaviruses comprise a diverse group of enveloped positive-strand RNA viruses that are responsible for several human diseases, most notably the severe acute respiratory syndrome (SARS) which emerged in 2003. Perturbation of the host cell cycle regulation is a characteristic feature of infections by many DNA and RNA-viruses, including Corona-virus infectious bronchitis virus (IBV) (Xu L. H. et al.: Coronavirus Infection Induces DNA Replication Stress Partly through Interaction of Its Nonstructural Protein 13 with the p125 Subunit of DNA Polymerase J Biol Chem 286: 39546-39559, 2011). IBV infection was shown to induce cell cycle arrest at both S and G2/M phases for the enhancement of viral replication and progeny production. Xu et al. have shown that activation of the cellular DNA damage response is one of the key mechanisms exploited by Coronavirus to induce cell cycle arrest.

The DNA damage response is mediated by members of the PIKK (phosphatidylinositol-3-kinase-like protein kinase) family of serine/threonine kinases including ATM (ataxia telangiectasia mutated), ATR (ataxia telangiectasia and Rad3 related), and DNA-PK (DNA-dependent protein kinase) (Luftig et al., Annu. Rev. Vir. 2014. 1:605-25). Both ATM and ATR are activated by DNA damage and DNA replication stress, but their roles in the DNA-damage response are different and not redundant. ATM and ATR often work together to signal DNA damage and regulate downstream processes. ATM is primarily activated by double-stranded DNA breaks (DSBs), while ATR is activated by single stranded DNA during the S phase of the cell cycle.

Xu et al (ibid.) showed that ATR-signaling was activated in IBV-infected H1299 as well as Vero cells. Suppression of the ATR kinase activity by chemical inhibitors and siRNA-mediated knockdown of ATR reduced IBV-induced ATR signaling and inhibited the replication of IBV. On the contrary, ATM pathway activation was not observed and ATM inhibitors did not reduce IBV replication.

Luftig et al. (ibid) reviewed the general relationship between the DNA damage response and viruses, albeit without specific reference to coronaviruses, pointing out that virus-induced DNA damage response activation can be broad and include activation of ATM, DNA-PK and ATR protein kinases.

In light of the antiviral activity observed with the potent and selective ATM inhibitors as shown herein, it is hypothesized that compounds of the invention advantageously interfere with the DNA damage response and virus replication. It is conceivable that the ATM inhibitors inhibit the coronavirus induced cell arrest and/or the replication of the coronavirus in the host cell by inhibiting the virus induced activation of cellular DNA damage response. Whatever the exact mechanism of action for the antiviral properties of the compounds of the invention, it is proposed that administration thereof may have one or more clinical benefits, as described further herein.

“COVID-19” is the name of the disease which is caused by a SARS-CoV-2 infection. While care was taken to describe both the infection and disease with accurate terminology, “COVID-19” and “SARS-CoV-2 infection” are meant to be equivalent terms.

As of the writing of this application, the determination and characteristics of the severity of COVID-19 patients/symptoms has not been definitively established. However, in the context of this invention, “mild to moderate” COVID-19 occurs when the subject presents as asymptomatic or with less severe clinical symptoms (e.g., low grade or no fever (<39.1° C.), cough, mild to moderate discomfort) with no evidence of pneumonia, and generally does not require medical attention. When “moderate to severe” infection is referred to, generally patients present with more severe clinical symptoms (e.g., fever >39.1° C., shortness of breath, persistent cough, pneumonia, etc.). As used herein “moderate to severe” infection typically requires medical intervention, including hospitalization. During the progression of disease, a subject can transition from “mild to moderate” to “moderate to severe” and back again in one course of bout of infection.

Treatment of COVID-19 using the methods of this invention include administration of an effective amount of an ATM inhibitor of the invention at any stage of the infection to prevent or reduce the symptoms associated therewith. Typically, subjects will be administered an effective amount of an ATM inhibitor of the invention after definitive diagnosis and presentation with symptoms consistent with a SARS-CoV2 infection, and administration will reduce the severity of the infection and/or prevent progression of the infection to a more severe state. The clinical benefits upon such administration is described in more detail in the sections below.

### 1. Compounds and Definitions

One embodiment is use of a first compound, an ATM inhibitor, according to the following formula:

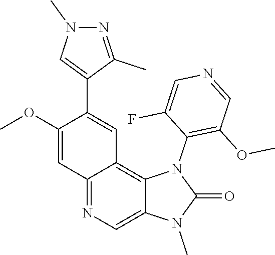

or a pharmaceutically acceptable salt thereof for the treatment of a viral infection.

The first compound respectively ATM inhibitor may also be referred to as 8-(1,3-dimethyl-1H-pyrazol-4-yl)-1-(3-fluoro-5-methoxy-pyridin-4-yl)-7-methoxy-3-methyl-1,3-dihydroimidazo[4,5-c]quinolin-2-one. It is disclosed and further characterized as Example 4 in WO2016/155844. In an exemplary embodiment, an axially chiral form of this first compound or pharmaceutically acceptable salt thereof is used, which is referred to as 8-(1,3-dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one and illustrated by the formula below (in the following also referred to as “NCE4”):

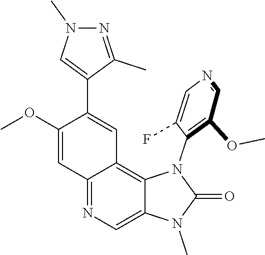

Any reference to the first compound or first ATM inhibitor in the following shall be read as including a reference to 8-(1,3-dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one.

One embodiment is use of a second compound, an ATM inhibitor, according to the following formula:

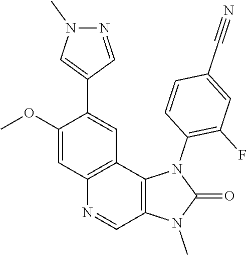 [SMILES: COc1cc2ncc3c(c2cc1-c1cnn(C)c1)n(-c1ccc(C#N)cc1F)c(=O)n3C]

or a pharmaceutically acceptable salt thereof for the treatment of a viral infection.

The second compound may also be designated 3-fluoro-4-[7-methoxy-3-methyl-8-(1-methyl-1H-pyrazol-4-yl)-2-oxo-2,3-dihydro-imidazo[4,5-c]quinolin-1-yl]-benzonitrile and is disclosed, including its synthesis, in WO2012/028233. It may, in the following, also be referred to as “NCE16”. Both first and second compounds are highly selective and potent inhibitors of ATM.

The above compounds may either be used in their free forms or as pharmaceutically acceptable salts. The free compounds may be converted into the associated acid-addition salt by reaction with an acid, for example by reaction of equivalent amounts of the base and the acid in an inert solvent, such as, for example, ethanol, and subsequent evaporation. Suitable acids for this reaction are, in particular, those which give physiologically acceptable salts, such as, for example, hydrogen halides (for example hydrogen chloride, hydrogen bromide or hydrogen iodide), other mineral acids and corresponding salts thereof (for example sulfate, nitrate or phosphate and the like), alkyl- and monoarylsulfonates (for example ethanedisulfonate (edisylate), toluenesulfonate, napthalene-2-sulfonate (napsylate), benzenesulfonate) and other organic acids and corresponding salts thereof (for example fumarate, oxalate, acetate, trifluoroacetate, tartrate, maleate, succinate, citrate, benzoate, salicylate, ascorbate) and the like.

Exemplary embodiments of pharmaceutically acceptable salts of the first compound or its atropisomer(s) comprise edisylate, fumarate and napsylate salts. Exemplary embodiments of pharmaceutically acceptable salts of the second compound comprise sulphate, maleate and oxalate, to name just a few examples.

Unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. In some embodiments, the compound comprises one or more deuterium atoms.

### 2. Uses, Formulation and Administration

The term “patient” or “subject”, as used herein, means an animal, preferably a human. However, “subject” can include companion animals such as dogs and cats. In one embodiment, the subject is an adult human patient. In another embodiment, the subject is a pediatric patient. Pediatric patients include any human which is under the age of 18 at the start of treatment. Adult patients include any human which is age 18 and above at the start of treatment. In one embodiment, the subject is a member of a high-risk group, such as being over 65 years of age, immunocompromised humans of any age, humans with chronic lung conditions (such as, asthma, COPD, cystic fibrosis, etc.), and humans with other co-morbidities. In one aspect of this embodiment, the other co-morbidity is obesity, diabetes, and/or hypertension.

Compositions of the present invention are administered orally, parenterally, by inhalation spray, topically, rectally, nasally, buccally, vaginally or via an implanted reservoir. Preferably, the compositions are administered orally. In one embodiment, an oral formulation (composition) of a compound of the invention is a tablet or capsule form. In another embodiment, the oral formulation is a solution or suspension which may be given to a subject in need thereof via mouth or nasogastric tube. Any oral formulations of the invention may be administered with or without food. In some embodiments, pharmaceutically acceptable compositions of this invention are administered without food. In other embodiments, pharmaceutically acceptable compositions of this invention are administered with food.

Pharmaceutically acceptable compositions of this invention are orally administered in any orally acceptable dosage form. Exemplary oral dosage forms are capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents are optionally also added.

The amount of compounds of the present invention that are optionally combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

In one embodiment, the total amount of ATM inhibitor administered to the subject in need thereof is between about 5 mg to about 1000 mg per day.

In one embodiment, the ATM inhibitor is administered in a total amount of 5 mg to 1 g per day, for instance between 10 and 750 mg per day, such as between 20 and 500 mg per day or between 50 and 500 mg per day. In one embodiment, the atropisomer of the first compound (“NCE4”) is administered in a total amount of 25 to 350 mg per day. In one embodiment, the second compound is administered in a total amount of 150 to 480 mg per day.

In another embodiment, the ATM inhibitor is administered once a day. In another aspect of this embodiment, the ATM inhibitor is administered twice a day.

In any of the above embodiments, the ATM inhibitor is administered for a period of about 7 days to about 28 days. In one aspect of any of the above embodiments, the ATM inhibitor is administered for about 14 days.

In one embodiment of the invention, the subject is suffering from COVID-19 pneumonia. In one embodiment of this invention, the subject is suffering from one or more symptoms selected from chest congestion, cough, blood oxygen saturation (SpO₂) levels below 94%, shortness of breath, difficulty breathing, fever, chills, repeated shaking with chills, muscle pain and/or weakness, headache, sore throat and/or new loss of taste or smell.

In one embodiment, the subject is suffering from a hyperinflammatory host immune response to a SARS-CoV-2 infection. In one aspect of this embodiment, the hyperinflammatory host immune response is associated with one or more clinical indications selected from 1) reduced levels of lymphocytes, especially natural killer (NK) cells in peripheral blood; 2) high levels of inflammatory parameters (eg, C reactive protein [CRP], ferritin, d-dimer), and pro-inflammatory cytokines (eg, IL-6, TNF-alpha, IL-8, and/or IL-1beta; 3) a deteriorating immune system demonstrated by lymphocytopenia and/or atrophy of the spleen and lymph nodes, along with reduced lymphocytes in lymphoid organs; 4) dysfunction of the lung physiology represented by lung lesions infiltrated with monocytes, macrophages, and/or neutrophils, but minimal lymphocytes infiltration resulting in decreased oxygenation of the blood; 5) acute respiratory distress syndrome (ARDS); 6) vasculitis; 7) encephalitis, Guillain-Barre syndrome, and other neurologic disorders; 8) kidney dysfunction and kidney failure; 9) hypercoagulability such as arterial thromboses; and 10) or any combination of above resulting in end-organ damage and death.

In one embodiment, the subject with COVID-19 is a pediatric patient suffering from vasculitis, including Kawasaki disease (i.e., Kawasaki syndrome) and Kawasaki-like disease.

In one embodiment of the invention, the subject is being treated inpatient in a hospital setting. In another embodiment, the subject is being treated in an outpatient setting. In one aspect of the preceding embodiments, the subject may continue administration of the ATM inhibitor after being transitioned from being treated from an inpatient hospital setting to an outpatient setting.

In one embodiment, the administration of the ATM inhibitor results in one or more clinical benefit. In one aspect of this embodiment, the one or more clinical benefit is selected from the group comprising: reduction of duration of a hospital stay, reduction of the duration of time in the Intensive Care Unit (ICU), reduction in the likelihood of the subject being admitted to an ICU, reduction in the rate of mortality, reduction in the likelihood of kidney failure requiring dialysis, reduction in the likelihood of being put on non-invasive or invasive mechanical ventilation, reduction of the time to recovery, reduction in the likelihood supplemental oxygen will be needed, improvement or normalization in the peripheral capillary oxygen saturation (SpO₂ levels) without mechanical intervention, reduction of severity of the pneumonia as determined by chest imaging (eg, CT or chest X ray), reduction in the cytokine production, reduction of the severity of acute respiratory distress syndrome (ARDS), reduction in the likelihood of developing ARDS, clinical resolution of the COVID-19 pneumonia, and improvement of the PaO₂/FiO₂ ratio.

In another embodiment, the one or more clinical benefits includes the improvement or normalization in the peripheral capillary oxygen saturation (SpO₂ levels) in the subject without mechanical ventilation or extracorporeal membrane oxygenation.

In a further embodiment, the one of more clinical benefits is reduction in the likelihood of being hospitalized, reduction in the likelihood of ICU admission, reduction in the likelihood being intubated (invasive mechanical ventilation), reduction in the likelihood supplemental oxygen will be needed, reduction in the length of hospital stay, reduction in the likelihood of mortality, and/or a reduction in likelihood of relapse, including the likelihood of rehospitalization.

The invention also provides a method of treating a viral infection in a subject in need thereof comprising administering an effective amount of a compound of the invention to the subject. An amount effective to treat or inhibit a viral infection is an amount that will cause a reduction in one or more of the manifestations of viral infection, such as viral lesions, viral load, rate of virus production, and mortality as compared to untreated control subjects.

One embodiment of the invention is a method of treating a coronavirus infection in a subject in need thereof, comprising administering an effective amount of an ATM inhibitor, or a pharmaceutically acceptable salt thereof, to the subject. In one aspect of this embodiment, the subject is infected with SARS-CoV-2. In another aspect of this embodiment, the administration of the ATM inhibitor results in the reduction of the viral load in the subject.

In one embodiment, the ATM inhibitor is administered prior to COVID-19 pneumonia developing. In another embodiment, the subject has a mild to moderate SARS-CoV-2 infection. In a further embodiment, the subject is asymptomatic at the start of the administration regimen. In another embodiment, the subject has had known contact with a patient who has been diagnosed with a SARS-CoV-2 infection. In an additional embodiment, the subject begins administration of the ATM inhibitor prior to being formally diagnosed with COVID-19.

One embodiment is a method of treating a subject with COVID-19 comprising administration of an effective amount of an ATM inhibitor to the subject. In one aspect of this embodiment, the subject has been previously vaccinated with a SARS-CoV-2 vaccine and develops vaccine-related exacerbation of infection, for example, an antibody-dependent enhancement or related antibody-mediated mechanisms of vaccine/antibody-related exacerbation.

In any of the above embodiments, the administration of the ATM inhibitor results in one or more clinical benefits to the subject. In one aspect of this embodiment, the one or more clinical benefits is shortening the duration of infection, reduction of the likelihood of hospitalization, reduction in the likelihood of mortality, reduction in the likelihood of ICU admission, reduction in the likelihood of being placed on mechanical ventilation, reduction in the likelihood supplemental oxygen will be needed, and/or reduction in the length of hospital stay. In another aspect of this embodiment, the one or more clinical benefits is avoidance of a significant proinflammatory response. In a further aspect of this embodiment, the one or more clinical benefit is the failure of the subject to develop significant symptoms of COVID-19.

The compound(s) of the invention can be administered before or following an onset of SARS-CoV-2 infection, or after acute infection has been diagnosed in a subject. The aforementioned compounds and medical products of the inventive use are particularly used for the therapeutic treatment. A therapeutically relevant effect relieves to some extent one or more symptoms of a disorder, or returns to normality, either partially or completely, one or more physiological or biochemical parameters associated with or causative of a disease or pathological condition. Monitoring is considered as a kind of treatment provided that the compounds are administered in distinct intervals, e.g. in order to boost the response and eradicate the pathogens and/or symptoms of the disease. The methods of the invention can also be used to reduce the likelihood of developing a disorder or even prevent the initiation of disorders associated with COVID-19 in advance of the manifestation of mild to moderate disease, or to treat the arising and continuing symptoms of an acute infection.

Treatment of mild to moderate COVID-19 is typically done in an outpatient setting. Treatment of moderate to severe COVID-19 is typically done inpatient in a hospital setting. Additionally, treatment can continue in an outpatient setting after a subject has been discharged from the hospital.

The invention furthermore relates to a medicament comprising at least one compound according to the invention or a pharmaceutically salts thereof.

A “medicament” in the meaning of the invention is any agent in the field of medicine, which comprises one or more compounds of the invention or preparations thereof (e.g. a pharmaceutical composition or pharmaceutical formulation) and can be used in prophylaxis, therapy, follow-up or aftercare of patients who suffer from clinical symptoms and/or known exposure to COVID-19.

### Combination Treatment

In various embodiments, the ATM inhibitor may be administered alone or in combination with one or more additional therapeutic agents. A synergistic or augmented effect may be achieved by using more than one active ingredient in the pharmaceutical composition. The ATM inhibitor and one or more additional therapeutic agents can be used either simultaneously or sequentially.

In one embodiment, the ATM inhibitor is administered in combination with one or more additional therapeutic agents. In one aspect of this embodiment, the one or more additional therapeutic agents is selected from anti-inflammatories, antibiotics, anti-coagulants, antiparasitic agent, antiplatelet agents and dual antiplatelet therapy, angiotensin converting enzyme (ACE) inhibitors, angiotensin II receptor blockers, beta-blockers, statins and other combination cholesterol lowering agents, specific cytokine inhibitors, complement inhibitors, anti-VEGF treatments, JAK inhibitors, immunomodulators, anti-inflammasome therapies, sphingosine-1 phosphate receptors binders, N-methyl-d-aspartate (NDMA) receptor glutamate receptor antagonists, corticosteroids, Granulocyte-macrophage colony-stimulating factor (GM-CSF), anti-GM-CSF, interferons, angiotensin receptor-neprilysin inhibitors, calcium channel blockers, vasodilators, diuretics, muscle relaxants, and antiviral medications.

In one embodiment, the ATM inhibitor is administered in combination with an antiviral agent. In one aspect of this embodiment, the antiviral agent is remdesivir. In another aspect of this embodiment, the antiviral agent is lopinavir-ritonavir, alone or in combination with ribavirin and interferon-beta.

In one embodiment, the ATM inhibitor is administered in combination with a broad-spectrum antibiotic.

In one embodiment, the ATM inhibitor is administered in combination with chloroquine or hydroxychloroquine. In one aspect of this embodiment, the ATM inhibitor is further combined with azithromycin.

In one embodiment, the ATM inhibitor is administered in combination with interferon-1-beta (Rebif®).

In one embodiment, the ATM inhibitor is administered in combination with one or more additional therapeutic agents selected from hydroxychloroquine, chloroquine, ivermectin, tranexamic acid, nafamostat, virazole, ribavirin, lopinavir/ritonavir, favipiravir, arbidol, leronlimab, interferon beta-la, interferon beta-1b, beta-interferon, azithromycin, nitrazoxamide, lovastatin, clazakizumab, adalimumab, etanercept, golimumab, infliximab, sarilumab, tocilizumab, anakinra, emapalumab, pirfenidone, belimumab, rituximab, ocrelizumab, anifrolumab, ravulizumab-cwvz, eculizumab, bevacizumab, heparin, enoxaparin, apremilast, coumadin, baricitinib, ruxolitinib, dapagliflozin, methotrexate, leflunomide, azathioprine, sulfasalazine, mycophenolate mofetil, colchicine, fingolimod, ifenprodil, prednisone, cortisol, dexamethasone, methylprednisolone, melatonin, otilimab, ATR-002, APN-01, camostat mesylate, brilacidin, IFX-1, PAX-1-001, BXT-25, NP-120, intravenous immunoglobulin (IVIG), and solnatide.

In one embodiment, the ATM inhibitor is administered in combination with one or more anti-inflammatory agent. In one aspect of this embodiment, the anti-inflammatory agent is selected from corticosteroids, steroids, COX-2 inhibitors, and non-steroidal anti-inflammatory drugs (NSAID). In one aspect of this embodiment, the anti-inflammatory agent is diclofenac, etodolac, fenoprofen, flurbirprofen, ibuprofen, indomethacin, meclofenamate, mefenamic acid, meloxicam, nabumetone, naproxen, oxaprozin, piroxicam, sulindac, tolmetin, celecoxib, prednisone, hydrocortisone, fludocortisone, bethamethasone, prednisolone, triamcinolone, methylprednisone, dexamethasone, fluticasone, and budesonide (alone or in combination with formoterol, salmeterol, or vilanterol).

In one embodiment, the ATM inhibitor is administered in combination with one or more immune modulators. In one aspect of this embodiment, the immune modulator is a calcineurin inhibitor, antimetabolite, or alkylating agent. In another aspect of this embodiment, the immune modulator is selected from azathioprine, mycophenolate mofetil, methotrexate, dapson, cyclosporine, cyclophosphamide, and the like.

In one embodiment, the ATM inhibitor is administered in combination with one or more antibiotics. In one aspect of this embodiment, the antibiotic is a broad-spectrum antibiotic. In another aspect of this embodiment, the antibiotic is a penicillin, anti-straphylococcal penicillin, cephalosporin, aminopenicillin (commonly administered with a betalactamase inhibitor), monobactam, quinoline, aminoglycoside, lincosamide, macrolide, tetracycline, glycopeptide, antimetabolite or nitroimidazole. In a further aspect of this embodiment, the antibiotic is selected from penicillin G, oxacillin, amoxicillin, cefazolin, cephalexin, cephotetan, cefoxitin, ceftriazone, augmentin, amoxicillin, ampicillin (plus sulbactam), piperacillin (plus tazobactam), ertapenem, ciprofloxacin, imipenem, meropenem, levofloxacin, moxifloxacin, amikacin, clindamycin, azithromycin, doxycycline, vancomycin, Bactrim, and metronidazole.

In one embodiment, the ATM inhibitor is administered in combination with one or more anti-coagulants. In one aspect of this embodiment, the anti-coagulant is selected from apixaban, dabigatran, edoxaban, heparin, rivaroxaban, and warfarin.

In one embodiment, the ATM inhibitor is administered in combination with one or more antiplatelet agents and/or dual antiplatelet therapy. In one aspect of this embodiment, the antiplatelet agent and/or dual antiplatelet therapy is selected from aspirin, clopidogrel, dipyridamole, prasugrel, and ticagrelor.

In one embodiment, the ATM inhibitor is administered in combination with one or more ACE inhibitors. In one aspect of this embodiment, the ACE inhibitor is selected from benazepril, captopril, enalapril, fosinopril, lisinopril, moexipril, perindopril, quinapril, ramipril and trandolapril.

In one embodiment, the ATM inhibitor is administered in combination with one or more angiotensin II receptor blockers. In one aspect of this embodiment, the angiotensin II receptor blocker is selected from azilsartan, candesartan, eprosartan, irbesartan, losartan, Olmesartan, telmisartan, and valsartan.

In one embodiment, the ATM inhibitor is administered in combination with one or more beta-blockers. In one aspect of this embodiment, the beta-blocker is selected from acebutolol, atenolol, betaxolol, bisoprolol/hydrochlorothiazide, bisoprolol, metoprolol, nadolol, propranolol, and sotalol.

In another embodiment, the ATM inhibitor is administered in combination with one or more alpha and beta-blocker. In one aspect of this embodiment, the alpha and beta-blocker is carvedilol or labetalol hydrochloride.

In one embodiment, the ATM inhibitor is administered in combination with one or more interferons.

In one embodiment, the ATM inhibitor is administered in combination with one or more angiotensin receptor-neprilysin inhibitors. In one aspect of this embodiment, the angiotensin receptor-neprilysin inhibitor is sacubitril/valsartan.

In one embodiment, the ATM inhibitor is administered in combination with one or more calcium channel blockers. In one aspect of this embodiment, the calcium channel blocker is selected from amlodipine, diltiazem, felodipine, nifedipine, nimodipine, nisoldipine, and verapamil.

In one embodiment, the ATM inhibitor is administered in combination with one or more vasodilators. In one aspect of this embodiment, the one or more vasodilator is selected from isosorbide dinitrate, isosorbide mononitrate, nitroglycerin, and minoxidil.

In one embodiment, the ATM inhibitor is administered in combination with one or more diuretics. In one aspect of this embodiment, the one or more diuretics is selected from acetazolamide, amiloride, bumetanide, chlorothiazide, chlorthalidone, furosemide, hydrochlorothiazide, indapamide, metolazone, spironolactone, and torsemide.

In one embodiment, the ATM inhibitor is administered in combination with one or more muscle relaxants. In one aspect of this embodiment, the muscle relaxant is an antispasmodic or antispastic. In another aspect of this embodiment, the one or more muscle relaxants is selected from carisoprodol, chlorzoxazone, cyclobenzaprine, metaxalone, methocarbamol, orphenadrine, tizanidine, baclofen, dantrolene, and diazepam.

In one embodiment, the ATM inhibitor is administered in combination with one or more antiviral medications. In one aspect of this embodiment, the antiviral medication is remdesivir.

In one embodiment, the ATM inhibitor is administered in combination with one or more additional therapeutic agents selected from antiparasitic drugs (including, but not limited to, hydroxychloroquine, chloroquine, ivermectin), antivirals (including, but not limited to, tranexamic acid, nafamostat, virazole [ribavirin], lopinavir/ritonavir, favipiravir, leronlimab, interferon beta-la, interferon beta-1b, beta-interferon), antibiotics with intracellular activities (including, but not limited to azithromycin, nitrazoxamide), statins and other combination cholesterol lowering and anti-inflammatory drugs (including, but not limited to, lovastatin), specific cytokine inhibitors (including, but not limited to, clazakizumab, adalimumab, etanercept, golimumab, infliximab, sarilumab, tocilizumab, anakinra, emapalumab, pirfenidone), complement inhibitors (including, but not limited to, ravulizumab-cwvz, eculizumab), anti-VEGF treatments (including, but not limited to, bevacizumab), anti-coagulants (including, but not limited to, heparin, enoxaparin, apremilast, coumadin), JAK inhibitors (including, but not limited to, baricitinib, ruxolitinib, dapagliflozin), anti-inflammasome therapies (including, but not limited to, colchicine), sphingosine-1 phosphate receptors binders (including, but not limited to, fingolimod), N-methyl-d-aspartate (NDMA) receptor glutamate receptor antagonists (including, but not limited to, ifenprodil), corticosteroids (including, but not limited to, prednisone, cortisol, dexamethasone, methylprednisolone), GM-CSF, anti-GM-CSF (otilimab), ATR-002, APN-01, camostat mesylate, arbidol, brilacidin, IFX-1, PAX-1-001, BXT-25, NP-120, intravenous immunoglobulin (IVIG), and solnatide.

In some embodiments, the combination of an ATM inhibitor with one or more additional therapeutic agents reduces the effective amount (including, but not limited to, dosage volume, dosage concentration, and/or total drug dose administered) of the ATM inhibitor and/or the one or more additional therapeutic agents administered to achieve the same result as compared to the effective amount administered when the ATM inhibitor or the additional therapeutic agent is administered alone. In some embodiments, the combination of an ATM inhibitor with the additional therapeutic agent reduces the total duration of treatment compared to administration of the additional therapeutic agent alone. In some embodiments, the combination of an ATM inhibitor with the additional therapeutic agent reduces the side effects associated with administration of the additional therapeutic agent alone. In some embodiments, the combination of an effective amount of the ATM inhibitor with the additional therapeutic agent is more efficacious compared to an effective amount of the ATM inhibitor or the additional therapeutic agent alone. In one embodiment, the combination of an effective amount of the ATM inhibitor with the one or more additional therapeutic agent results in one or more additional clinical benefits than administration of either agent alone.

As used herein, the terms “treatment,” “treat,” and “treating” refer to reversing, alleviating, delaying the onset of, or inhibiting the progress of a viral infection, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a known exposure to an infected person and/or in light of comorbidities which are predictors for severe disease, or other susceptibility factors).

### EXEMPLIFICATION

As described in the Examples below, in certain exemplary embodiments, compounds are prepared according to the following procedures.

### Example 1

The first compound is prepared in accordance with the procedure disclosed in WO 2016/155844, followed by separation of the atropisomers, as illustrated by the following reaction scheme:

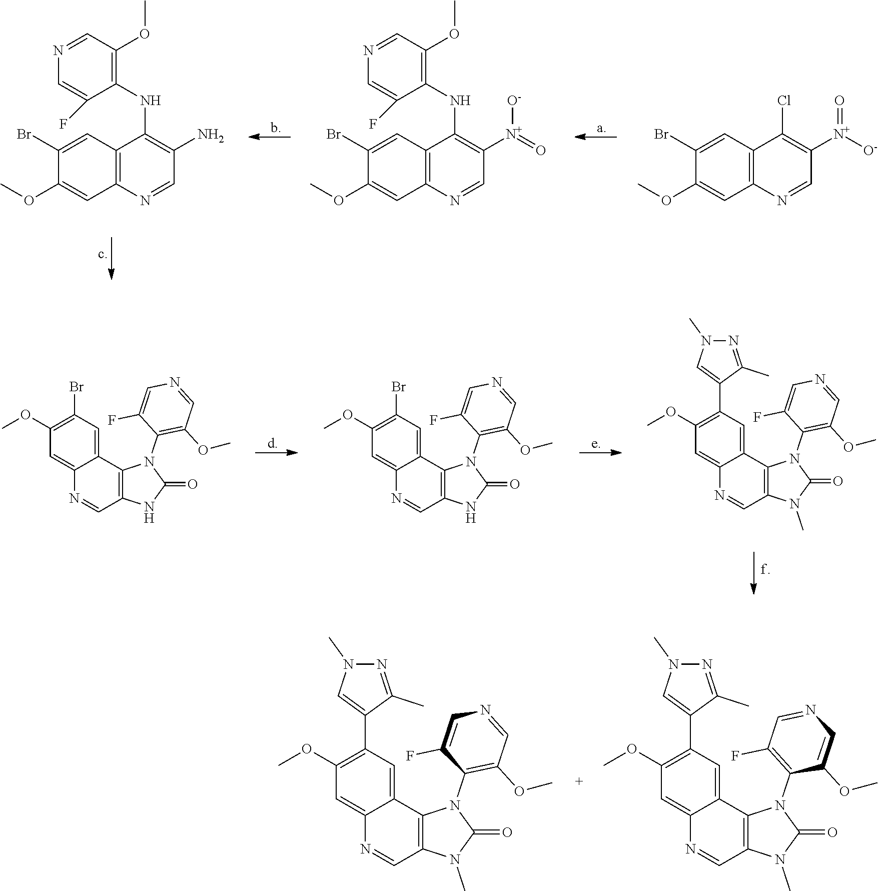

### a. Synthesis of 6-bromo-N-(3-fluoro-5-methoxy-4-pyridyl)-7-methoxy-3-nitro-quinolin-4-amine

Under a dry nitrogen atmosphere, a solution of 3-fluoro-5-methoxypyridin-4-amine (447 mg, 3.02 mmol) dissolved in N,N-dimethylformamide (5 mL) was provided. Then, sodium hydride (504 mg, 12.6 mmol, 60%) was added to the solution and stirring continued for 5 minutes at room temperature. 6-Bromo-4-chloro-7-methoxy-3-nitro-quinoline (800 mg, 2.52 mmol) was then added to the reaction mixture, followed by 15 minutes of stirring at room temperature, then by quenching of the reaction through addition of ice water (100 mL). The precipitate was filtered off, washed with ice water and dried to give 1.00 g (94%) 6-bromo-N-(3-fluoro-5-methoxy-4-pyridyl)-7-methoxy-3-nitro-quinolin-4-amine as a yellow solid.

### b. Synthesis of 6-bromo-N⁴-(3-fluoro-5-methoxy-4-pyridyl)-7-methoxy-quinoline-3,4-diamine

6-Bromo-N-(3-fluoro-5-methoxy-4-pyridyl)-7-methoxy-3-nitro-quinolin-4-amine (990 mg, 2.20 mmol) dissolved in methanol (100 mL) was provided under a protective nitrogen atmosphere. Then, Raney-Ni (100 mg, 1.17 mmol) was added to the solution, and the reaction mixture was stirred for 30 minutes under a hydrogen atmosphere at normal pressure. After introducing nitrogen, the suspension was filtered and the filtrate dried under vacuum. The filtrate was evaporated to dryness under vacuum. The residue was crystallized from a mixture of ethyl acetate/petroleum ether, yielding 0.86 g (99%) 6-bromo-N⁴-(3-fluoro-5-methoxy-4-pyridyl)-7-methoxy-quinoline-3,4-diamine as a yellow solid.

### c. Synthesis of 8-bromo-1-(3-fluoro-5-methoxy-4-pyridyl)-7-methoxy-3H-imidazo[4,5-c]quinolin-2-one

A solution of 6-bromo-N⁴-(3-fluoro-5-methoxy-4-pyridyl)-7-methoxy-quinoline-3,4-diamine (0.85 g, 2.20 mmol) dissolved in tetrahydrofuran (20 mL) was provided. Then, 1,1′-carbonyldiimidazole (1.84 g, 11.3 mmol) and Hünig's-base (1.46 g, 11.3 mmol) were added. The reaction mixture was heated to 40° C. and stirred for 16 hours. The reaction was then quenched by the addition of ice water (200 mL). The precipitate was filtered off, washed with ice water and dried to give 0.87 g (94%) 8-bromo-1-(3-fluoro-5-methoxy-4-pyridyl)-7-methoxy-3H-imidazo[4,5-c]quinolin-2-one as a light yellow solid.

### d. Synthesis of 8-bromo-1-(3-fluoro-5-methoxy-4-pyridyl)-7-methoxy-3-methyl-imidazo[4,5-c]quinolin-2-one

In a dry protective nitrogen gas atmosphere, 8-bromo-1-(3-fluoro-5-methoxy-4-pyridyl)-7-methoxy-3H-imidazo[4,5-c]quinolin-2-one (0.86 g, 1.94 mmol) dissolved in N,N-dimethylformamide (5 mL) was provided. Then, sodium hydride (388 mg, 9.71 mmol, 60%) and methyl iodide (2.76 g, 19.4 mmol) were added. The reaction mixture was stirred for 10 minutes at room temperature. Then the reaction was quenched by the addition of ice water (100 mL). The resulting precipitate was filtrated and dried under vacuum to give 0.70 g (80%) 8-bromo-1-(3-fluoro-5-methoxy-4-pyridyl)-7-methoxy-3-methyl-imidazo[4,5-c]quinolin-2-one as a light yellow solid.

### e. Synthesis of 1-(3-fluoro-5-methoxy-4-pyridyl)-7-methoxy-3-methyl-8-(1,3-dimethylpyrazol-4-yl)imidazo[4,5-c]quinolin-2-one

Under an argon inert gas atmosphere in closed equipment 8-bromo-1-(3-fluoro-5-methoxy-4-pyridyl)-7-methoxy-3-methyl-imidazo[4,5-c]quinolin-2-one (150 mg, 0.33 mmol), 1-3-dimethyl-4-(tetramethyl-1,3,2-dioxaborolan-2-yl)-1H-pyrazole (88.4 mg, 0.40 mmol), Pd(PPh₃)₄ (76.6 mg, 0.07 mmol) and potassium carbonate (91.6 mg, 0.66 mmol) in 1,4-dioxane (15 mL) and water (5 mL) were provided. The reaction mixture was heated to 80° C. with stirring for 2 hours. This was followed by cooling to room temperature and reducing the reaction mixture to dryness under vacuum. The residue was chromatographically purified using silica (ethyl acetate/methanol=97:3, parts by volume). The eluate was reduced to dryness and the resulting raw product purified by means or preparative RP-HPLC (water/acetonitrile). After reducing the product fractions, 1-(3-fluoro-5-methoxy-4-pyridyl)-7-methoxy-3-methyl-8-(1,3-dimethylpyrazol-4-yl)imidazo[4,5-c]quinolin-2-one (70 mg, 47%) was obtained as a colourless solid.

### f. Separation of 8-(1,3-dimethyl-1H-pyrazol-4-yl)-1-(Ra)-(3-fluoro-5-methoxy-pyridin-4-yl)-7-methoxy-3-methyl-1,3-dihydro-imidazo[4,5-c]quinolin-2-one and 8-(1,3-dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one

1-(3-fluoro-5-methoxy-4-pyridyl)-7-methoxy-3-methyl-8-(1,3-methylpyrazol-4-yl)imidazo[4,5-c]quinolin-2-one (50.0 mg, 0.11 mmol) as obtained above was separated via chiral HPLC using SFC. The substance was applied to chiral column Lux Cellulose-2 and separated at a flow of 5 mL/min with CO₂/2-propanol+0.5% diethylamine (75:25) as the solvent and using detection at a wavelength of 240 nm. Reducing the product fractions at reduced pressure yielded 8-(1,3-Dimethyl-1H-pyrazol-4-yl)-1-(Ra)-(3-fluoro-5-methoxy-pyridin-4-yl)-7-methoxy-3-methyl-1,3-dihydro-imidazo[4,5-c]quinolin-2-one (25.0 mg, 50%) and 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one) (22.1 mg, 44%), both as colourless solids.

The starting compounds for the above reactions are readily obtainable, for instance as shown below:

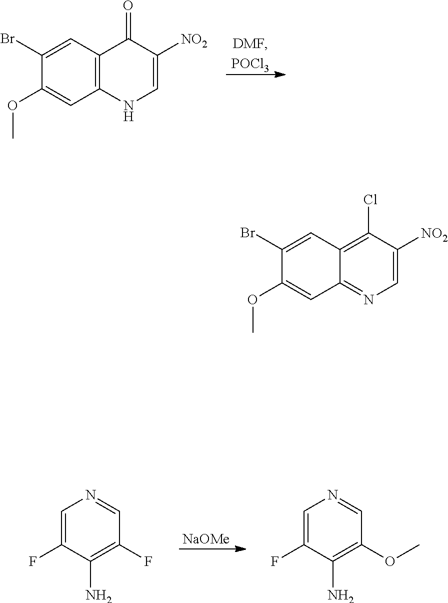

The atropisomers of the first compound can be isolated using chromatography on a chiral stationary phase (see, e.g., Chiral Liquid Chromatography; W. J. Lough, Ed. Chapman and Hall, New York, (1989); Okamoto, “Optical resolution of dihydropyridine enantiomers by high-performance liquid chromatography using phenylcarbamates of polysaccharides as a chiral stationary phase”, J. of Chromatogr. 513:375-378, (1990)). The atropisomers can be isolated by chromatography on chiral stationary phase, for example, a Chiralpak IC column (5 mm, 150×4.6 mm I.D.) e.g., using isocratic elution with a mobile phase containing: H₂O/ACN 50/50 v/v (ACN: acetonitrile; v: volume). A suitable chromatogram may be obtained using the following conditions: Column and elution as mentioned above, flow 1.00 ml/min; UV @ 260 nm; T_{c} and T_{S}: 25±5° C., S_{conc} 0.20 mg/ml; injected volume 10 ml.

As an alternative to the SFC conditions mentioned above, preparative supercritical fluid chromatography may be used, involving for instance: Chiralpak AS-H (20 mm×250 mm, 5 μm) column; isocratic elution (20:80 ethanol:CO₂ with 0.1% v/v NH₃), BPR (back-pressure reg.): about 100 bar above atmospheric pressure; a column temperature of 40° C., a flow rate of 50 ml/min, an injection volume of 2500 μl (125 mg) and a detector wavelength of 265 nm, with the (Sa)-atropisomer eluting second (after the (Ra)atropisomer)).

For the analysis of the purity of the respective atropisomers, again, SFC may be applied, for instance using the following set-up: Chiralpak AS-H (4.6 mm×250 mm, 5 μm) column; isocratic elution (20:80 ethanol:CO₂ with 0.1% v/v NH₃), BPR (back-pressure reg.): about 125 bar above atmospheric pressure; a column temperature of 40° C., a flow rate of 4 ml/min, an injection volume of 1 μl and a detector wavelength of 260 nm.

The atropisomers of the first compound may also be isolated through preparation of chiral salts, for instance using dibenzoyl-L-tartaric acid, as illustrated in the scheme below:

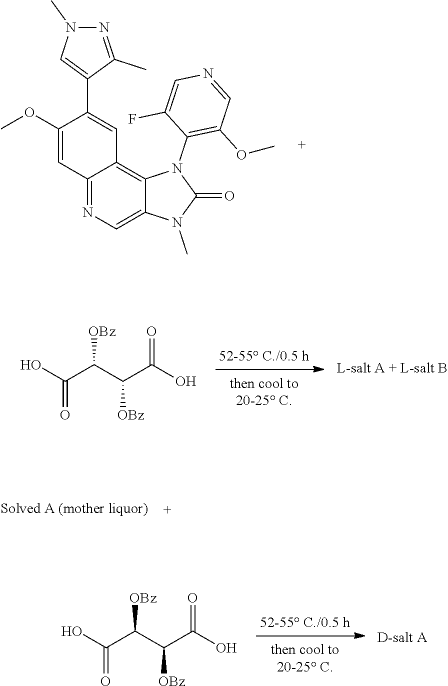

The second compound, or salt thereof, is prepared in accordance with the disclosure in WO2012/028233.

### Example 2: Antiviral Testing of Compounds

Calu-3 cells were seeded on two 384 well plates. Plate 1 contained compounds plus virus SARS-CoV2/ZG/297-20 Passage 6 0.05 multiplicity of infection and Plate 2 contained compounds only. For each well, 15,000 Calu-3 cells were seeded in 50 μL/well in full growth medium (EMEM, 10% FCS, 1% Pen/strep). The cells were grown for 48 hours at 37° C. and 5% CO₂. After this time, the medium in both plates was changed and fresh medium was added to each well.

On plate 1: 5 μL of each compound with respective concentrations were added to the specified wells in duplicates for 1 hour, and were infected afterwards with SARS-Cov-2 in an MOI of 0.05. The final volume of each well contained 5 μL compound, 5 μL virus (diluted and amount adjusted to 0.05 MOI), and 40 μL EMEM full medium for a total of 50 μL per well. The plate was monitored by Incucyte microscopy after virus addition at 2 h intervals, for a total observation time of 120 hours.

Viability of cells determined with Cell Glo reagent (Promega); 50 μL reagent was added to each well, incubated at RT in dark for 10 min, then the luminescence was measured with the Biotek plate reader.

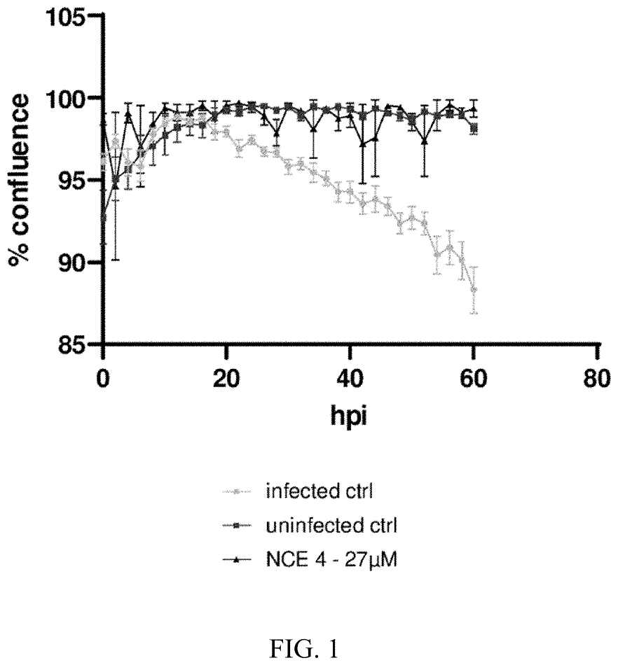

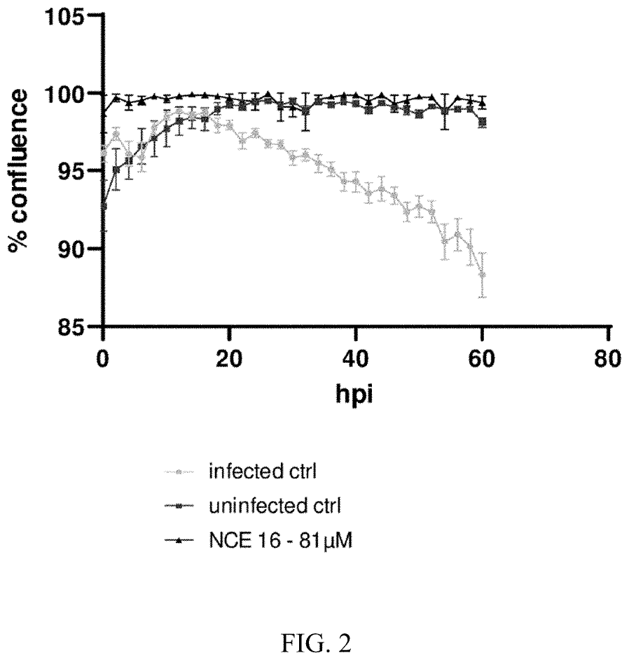

As apparent from FIGS. 1 and 2, both the first compound (more specifically (Sa) atropisomer thereof, “NCE4”) and the second compound (“NCE16”) lead to a significant retainment or improvement of the confluence of the cells as compared to the infected cells, with the level of confluence being about equal to the level of uninfected cells. The results shown in FIGS. 1 and 2 were reproducible.

1: A method of treating a coronavirus infection in a subject in need thereof, comprising:
administering an effective amount of an ataxia telangiectasia mutated kinase (ATM) inhibitor, or a pharmaceutically acceptable salt thereof, to the subject.

2: The method of claim 1, wherein the coronavirus causes a SARS or MERS infection.

3: The method of claim 1, wherein the coronavirus causes a SARS-CoV-1 or SARS-CoV-2 or MERS-CoV infection.

4: The method of claim 1, wherein the coronavirus is SARS-CoV-2.

5: The method of claim 1, wherein the ATM inhibitor is selected from the group consisting of 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one, 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, 3-fluoro-4-[7-methoxy-3-methyl-8-(1-methyl-1H-pyrazol-4-yl)-2-oxo-2,3-dihydro-imidazo[4,5-c]quinolin-1-yl]-benzonitrile, and pharmaceutically acceptable salts thereof.

6: The method of claim 1, wherein the administration of the ATM inhibitor results in viral load in the subject.

7-8. (canceled)

9: The method of claim 1, wherein the subject has a mild to moderate SARS-CoV-2 infection.

10-13. (canceled)

14: The method of claim 1, wherein the administration of the ATM inhibitor results in one or more clinical benefits.

15: The method of claim 14, wherein the one or more clinical benefits is selected from: shortening a duration of infection, reduction of a likelihood of hospitalization, reduction in a likelihood of mortality, reduction in a likelihood of intensive care unit (ICU) admission, reduction in likelihood being placed on mechanical ventilation, reduction in a likelihood supplemental oxygen will be needed, and/or reduction in length of hospital stay.

16. (canceled)

17: The method of claim 1, further comprising administration of one or more additional therapeutic agent.

18: The method of claim 17, wherein the one or more additional therapeutic agents is selected from an antiinflammatory, an antibiotic, an anti-coagulant, an antiparasitic agent, an antiplatelet agent and dual antiplatelet therapy, an angiotensin converting enzyme (ACE) inhibitor, an angiotensin 11 receptor blocker, a beta-blocker, a statin and other combination cholesterol lowering agent, a specific cytokine inhibitor, a complement inhibitor, an anti-VEGF treatment, an immunomodulator, an anti-inflammasome therapy, a sphingosine-1 phosphate receptor binder, an N-methyl-d-aspartate (NDMA) receptor glutamate receptor antagonist, a corticosteroid, a Granulocyte-macrophage colony-stimulating factor (GM-CSF), anti-GM-CSF, an interferon, an angiotensin receptor-neprilysin inhibitor, calcium channel blocker, a vasodilator, diuretic, a muscle relaxant, and an antiviral medication.

19: The method of claim 17, wherein the one or more additional therapeutic agents is an antiviral medication.

20: The method of claim 17, wherein the one or more additional therapeutic agents is remdesivir.

21-26. (canceled)

27: The method of claim 1, wherein the ATM inhibitor is administered daily.

28: The method of claim 1, wherein a total amount of ATM inhibitor administered is between about 20 mg and about 500 mg per day.

29: The method of claim 1, wherein the ATM inhibitor is administered for about 7 days to about 21 days.

30: The method of claim 1, wherein the ATM inhibitor is administered via oral administration.
